(19) United States
(12) Patent Application Publication
(10) Pub. No.: US 2022/0196552 A1
(43) Pub. Date: Jun. 23, 2022

(54) STANDARDIZATION OF MERFISH IMAGING SYSTEMS

(71) Applicant: Applied Materials, Inc., Santa Clara, CA (US)

(72) Inventor: Mehta Kalpesh Badreshkumar, Singapore (SG)

(73) Assignee: Applied Materials, Inc., Santa Clara, CA (US)

(21) Appl. No.: 17/557,522

(22) Filed: Dec. 21, 2021

Related U.S. Application Data
(60) Provisional application No. 63/128,747, filed on Dec. 21, 2020.

(51) Int. Cl. G01N 21/64; C12Q 1/6841

(52) U.S. Cl. CPC G01N 21/6458; G01N 2021/6478; G01N 21/6428; C12Q 1/6841

(57) ABSTRACT

Standardizing MERFISH imaging system provides a method to standardize a fluorescence microscope for a MERFISH analysis, the fluorescence microscope includes an excitation focus lens assembly and a light source. The method includes determining a roll-off value for the fluorescence microscope and adjusting the roll-off value of the fluorescence microscope to be 65% or lower by controlling a distance between the excitation focus lens assembly and the light source.

### CROSS-REFERENCE TO RELATED APPLICATIONS

This application claims priority to U.S. Provisional Application No. 63/128,747, filed Dec. 21, 2020, the entire disclosure of which is hereby incorporated by reference herein.

### TECHNICAL FIELD

The present disclosure generally relates to apparatus and methods for multiplex error robust fluorescence in-situ hybridization (MERFISH). In particular, embodiments of the disclosure are directed to methods and apparatus for standardizing MERFISH imaging systems.

### BACKGROUND

Fluorescence microscopy has become an important tool in life science research. With advancement in the field, the precision and usefulness of fluorescence microscopy has increased. With this advancement, instrumentation and analysis processes have become more complex and more susceptible to error.

Fluorescence microscopy can be largely divided into two main groups based on the applications: 1) qualitative microscopy for identification, structure elucidation and spatial distribution; and 2) quantitative microscopy to evaluate the amount of the fluorescing species is present. For qualitative and quantitative analyses, there are numerous parameters that can be used to define the performance of an imaging system.

Multiplexed Error-Robust Fluorescence In-Situ Hybridization, also known as MERFISH, is a recently developed technique. MERFISH allows for the quantification and determination of the spatial distribution of RNA inside a cell. Current MERFISH imaging systems lack quality control parameters. These parameters are used to define a stable operating range for the system. Without such parameters, it is difficult to qualify the imaging system (i.e., assess the health of the imaging system), determine system stability and/or compare the performance across the different imaging systems.

Therefore, there is a need in the art for apparatus and methods to define the stable operating range for an imaging system.

### SUMMARY

One aspect of the present disclosure pertains to methods of fluorescence image acquisition. A roll-off value is determined for a fluorescence microscope comprising a light source and an excitation focus lens assembly. The roll-off value of the fluorescence microscope is adjusted to be within a range of 31% to 65%. A first excitation wavelength and first emission wavelength is selected for a first fluorophore probe. A sample is hybridized with the first fluorophore probe. An image of the same is acquired using the fluorescence microscope configured with a first focal plane, the image comprising fluorescently emitted light from the sample.

Additional embodiments of the disclosure are directed to methods of fluorescence image acquisition, the methods comprising: determining a roll-off value for a fluorescence microscope, the fluorescence microscope comprises a light source and an excitation focus lens assembly; adjusting the roll-off value of the fluorescence microscope to be within a range of 31% to 65%, the roll-off value adjusted by controlling a distance between the light source and the excitation focus lens assembly; and quantitatively analyzing a sample using a plurality of fluorophore probes to generate a spatial distribution for each fluorophore probe.

Further embodiments of the disclosure are directed to fluorescence microscopes comprising a sample stage configured to hold a sample; a variable wavelength excitation light source directed at the sample stage; an excitation focus lens assembly configured to focus a light from the light source on the sample; a detector configured to detect light emitted from the sample; and a controller configured to adjust a roll-off value for the microscope to be in the range of 31% to 65%.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 shows a schematic representation of a MERFISH imaging system according to one or more embodiment of the disclosure; and.

FIG. 2 shows a flowchart of a process in accordance with one or more embodiment of the disclosure.

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

One or more embodiments of the disclosure advantageously provide one or more quality control parameter(s) to establish a stable baseline for a MERFISH imaging system. Some embodiments advantageously provide methods to measure quality control parameters to establish a stable baseline for a MERFISH imaging system.

Current quality control (QC) parameters for fluorescence imaging systems are not extendable to MERFISH assays. In some embodiments, MERFISH QC parameters are estimated by combining system measurements and information from biological sample analysis. For example, illumination power density is one of the parameters that can be used for standardization. In high resolution systems, including MERFISH imaging systems, it is difficult to measure the illumination power density (power/pixel) directly. Accordingly, one or more embodiment of the disclosure provides methods to define a stable operating range for the MERFISH imaging system by estimating power density.

In some embodiments, information from MERFISH analysis carried out on biological samples and imaging system parameters measured on the same system are combined to define stable operating range for the system. In one or more embodiments of the disclosure, the parameters defined allow for system qualification and/or determination of whether a particular system is operating within a stable range or requires maintenance. In one or more embodiments, the parameters allow for comparison of the performance across different MERFISH imaging systems.

As used in this specification and the appended claims, the term “standardization” refers to a general process of making one or more system function within a pre-determined stable operating range.

As used in this specification and the appended claims, a “fluorescence probe” refers to a composition comprising a fluorescent molecule that can produce fluorescence upon excitation by light having a pre-determined excitation wavelength. In some embodiments, the fluorescence probe comprises a polynucleotide conjugated to the fluorescence molecule.

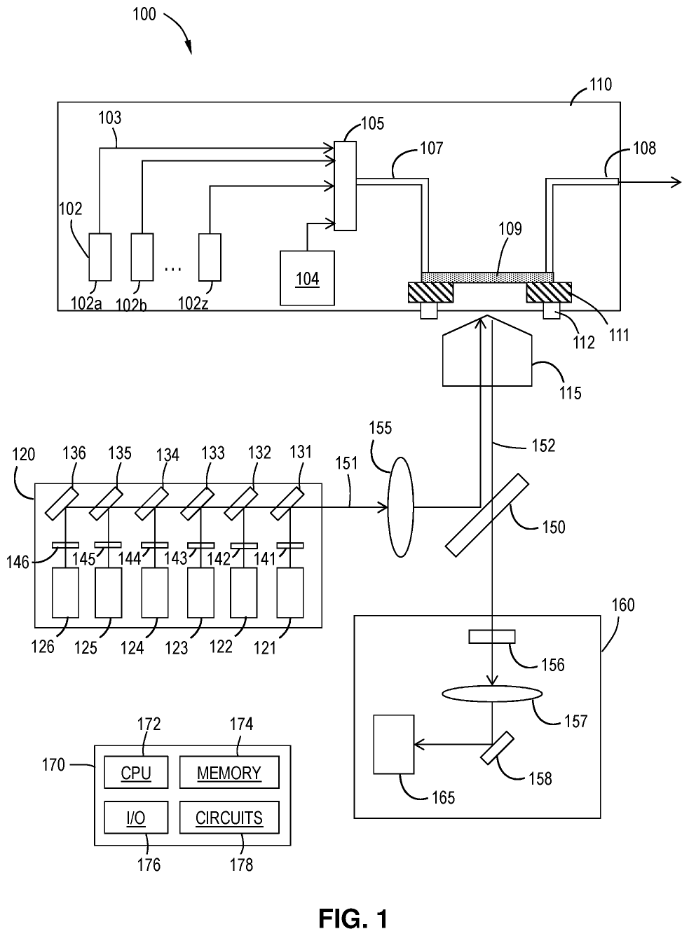

Generally, a fluorescence microscope includes an excitation light source configured to illuminate a target sample at an excitation wavelength, an emission filter configured to pass fluorescing light of a predetermined wavelength, and a detector configured to capture an image of illuminated target sample. FIG. 1 illustrates a schematic diagram of a Multiplexed Error-Robust Fluorescence In-Situ Hybridization (MERFISH) imaging system 100. MERFISH imaging system 100 comprises a flow cell assembly 110, a light source 120, a detector assembly 160 and a controller 170.

In some embodiments, the excitation light source comprises a xenon arc lamp or a mercury-vapor lamp. In some embodiments, the excitation light source further comprises an excitation filter.

In some embodiments, the excitation light source comprising a laser. In some embodiments, the laser has a wavelength in a range of from 400 nm to 850 nm, from 400 nm to 800 nm, from 400 nm to 750 nm, from 400 nm to 700 nm, from 400 nm to 650 nm, from 400 nm to 600 nm, from 400 nm to 550 nm, from 400 nm to 500 nm, from 400 nm to 450 nm, from 500 nm to 850 nm, from 500 nm to 800 nm, from 500 nm to 750 nm, from 500 nm to 700 nm, from 500 nm to 650 nm, from 500 nm to 600 nm, from 500 nm to 550 nm, from 600 nm to 850 nm, from 600 nm to 800 nm, from 600 nm to 750 nm, from 600 nm to 700 nm or from 600 nm to 650 nm.

In some embodiments, the fluorescence microscope comprises illumination optics. In some embodiments, the fluorescence microscope comprises optical filter.

The light source 120, also referred to as an excitation source, can by any suitable illumination system known to the skilled artisan. In the illustrated embodiment, the light source 120 comprises a plurality of individual laser sources 121, 122, 123, 124, 125, 126 in which each laser source 121, 122, 123, 124, 125, 126 emits light at a different wavelength (or frequency). The emitted light from the laser sources 121, 122, 123, 124, 125, 126 passes through a bandpass filter 141, 142, 143, 144, 145, 146, and reflects off of reflectors 131, 132, 133, 134, 135, 136 to exit the light source 120 as an excitation light beam 151. In the illustrated embodiment, there are six individual laser sources 121, 122, 123, 124, 125, 126 with associated bandpass filters and reflectors. However, the skilled artisan will recognize that there can be more or less than six laser sources. Also, the skilled artisan will recognize that the bandpass filters and/or reflectors are optional. In some embodiments, there are in the range of 2 to 10 laser sources, or in the range of 3 to 9 laser sources, or in the range of 4 to 8 laser sources or in the range of 5 to 7 laser sources, or 6 laser sources.

While the light source 120 illustrated in FIG. 1 shows a plurality of individual light sources, filters and reflectors, the skilled artisan will recognize that other sources configured to provide excitation energy are within the scope of the disclosure. For example, the light source 120 of some embodiments comprises a continuum source with a variable filter wheel. In one or more embodiments, the excitation light source comprises a xenon arc lamp, a mercury-vapor lamp, lasers and light emitting diode (LED).

The excitation light beam 151 exiting the light source 120 travels a distance and passes through an excitation focus lens assembly 155 which collimates or focuses the excitation light beam 151 onto dichroic cube 150 (also referred to as a dichroic mirror). In some embodiments, the excitation focus lens assembly comprises one or more collimating lenses. The excitation light beam 151 is directed off of the dichroic cube 150 to the objective 115 which is configured to focus light onto the sample flow cell 109. The objective 115 of some embodiments comprises an object lens and an aperture. In some embodiments, the stage 111 comprises an aperture. The optics illustrated—excitation focus lens assembly 155, dichroic cube 150 and objective 115—are shown outside of the light source 120 and the flow cell assembly 110. However, the skilled artisan will recognize that this is merely for descriptive purposes and should not be taken as limiting the scope of the disclosure. Any or all of the optics components shown, including the optics described below as part of the detector assembly 160 can be within the light source 120, detector assembly 160, flow cell assembly 110, or external to these components.

The flow cell assembly 110 comprises a sample flow cell 109 with an inlet 107 and an outlet 108 providing fluid communication with the sample flow cell 109 so that a fluid flowing through the inlet 107 passes into the sample flow cell 109, and out of the sample flow cell 109 through outlet 108.

One or more reservoirs 102 are in fluid communication with a pump 105 through lines 103. The one or more reservoirs 102 contain one or more fluorophore probe solutions 102a, 102b . . . 102z. In some embodiments, each of the fluorophore probe solutions 102a, 102b . . . 102z comprises a different fluorophore probe solution. As used in this manner, different fluorophore probe solutions have different fluorophore probe species. In some embodiments, there are greater than or equal to 2, 3, 4, 5, 6, 7, 8, 9, 10, 11, 12, 13, 14, 15, 16, 17, 18, 19 or 20 different fluorophore probe solutions. In some embodiments, there are in the range of 1 to 30 different fluorophore probe solutions, or in the range of 1 to 24 different fluorophore probe solutions, or in the range of 1 to 20 different fluorophore probe solutions, or in the range of 1 to 16 different fluorophore probe solutions, or in the range of 6 to 30 different fluorophore probe solutions, or in the range of 8 to 28 different fluorophore probe solutions, or in the range of 10 to 26 different fluorophore probe solutions, or in the range of 12 to 24 different fluorophore probe solutions, or in the range of 14 to 22 different fluorophore probe solutions, or in the range of 16 to 20 different fluorophore probe solutions.

The pump 105 of some embodiments selects one or more fluorophore probe solution from one or more of the reservoirs 102a-102z to flow through the sample flow cell 109. In some embodiments, the pump 105 is configured to flow fluorophore probe solution from one reservoir at a time.

The pump 105 of some embodiments is configured to rinse the sample flow cell 109 with a wash buffer solution from wash buffer reservoir 104 between flows of different fluorophore probe solutions. The wash buffer reservoir 104 can contain any suitable buffer solution depending on, for example, the sample being analyzed.

Excitation light beam 151 is directed onto the sample flow cell 109 through objective 115. The excitation light beam 151 excites the fluorophore probe in the sample flow cell 109. Fluorescing light 152 emitted from the sample flow cell 109 passes through dichroic cube 150 into detector assembly 160. In the illustrated embodiment, detector assembly 160 includes an emission filter 156, an emission focus lens 157 and mirror 158 onto detector 165. As discussed above, the emission filter 156, emission focus lens 157 and/or mirror 158 can be part of the detector assembly 160 or separate components from the detector assembly 160.

The detector 165 can be any suitable detector known to the skilled artisan. In some embodiments, the detector 165 comprises a charge-coupled device (CCD). In some embodiments, the detector 165 comprises a complementary metal-oxide semiconductor (CMOS) device.

The arrangement of optics in the embodiment illustrated in FIG. 1 is merely representative of one possible configuration and should not be taken as limiting the scope of the disclosure. The various optics including, mirrors, lenses, filters and dichroic cubes can be any suitable optics known to the skilled artisan arranged in any suitable configuration.

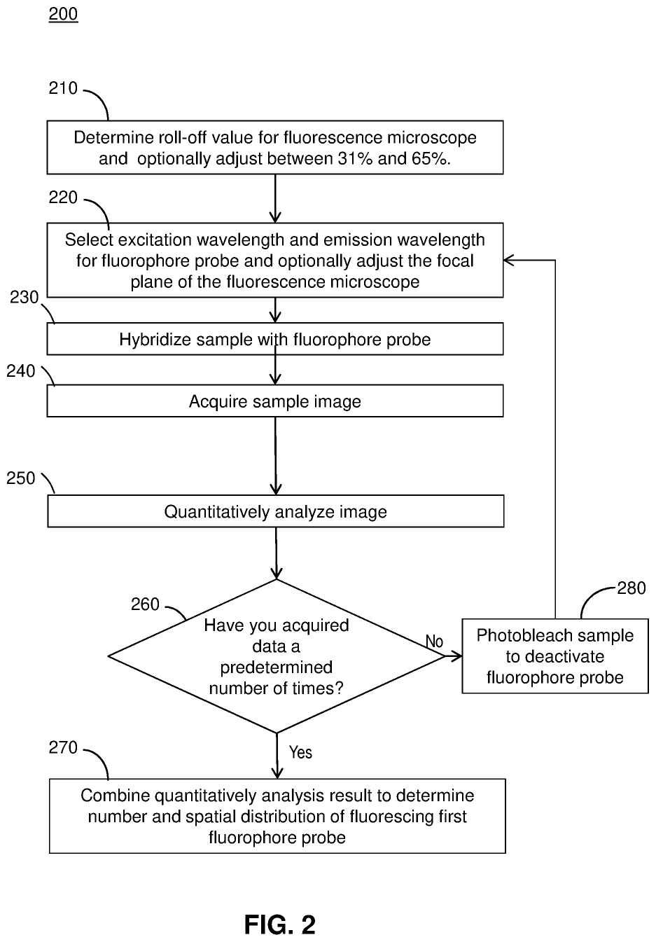

FIG. 2 illustrates a method 200 for a Multiplexed Error-Robust Fluorescence In-Situ Hybridization (MERFISH) process. Method 200 illustrates an exemplary process for standardizing a fluorescence microscope for obtaining reliable and reproducible MERFISH analysis.

An analysis of a MERFISH image uses detectable fluorescence signals from the illuminated sample. The fluorescence signal is directly proportional to the illumination power density. However, in conventional epi-illumination mode, there is a tradeoff between uniformity of the illumination power on the sample and power density. The illumination power density may affected by one or more of the light source (e.g., laser), illumination optics, optical filters and/or communication system. Further, system autofluorescence (i.e., naturally occurring light originating from or reflecting off of system components) also contributes to increasing noise levels. Illumination optics or optical filters may contribute to the system autofluorescence.

One or more embodiments of the disclosure provide methods to standardize the fluorescence microscope for MERFISH analysis. In particular, the disclosure provides a roll-off value of the fluorescence microscope as a way to adjust the illumination power density and thereby defining stable operating range for the MERFISH imaging system.

One aspect of the disclosure describes a method of fluorescence image acquisition. The method comprises determining a roll-off value for a fluorescence microscope, adjusting the roll-off value of the fluorescence microscope to 65% or less, selecting a first excitation wavelength and first emission wavelength for a first fluorophore probe, hybridizing a sample with the first fluorophore probe, and acquiring an image of the sample using the fluorescence microscope configured with a first focal plane, the image comprising fluorescently emitted light from the sample.

In one or more embodiments, the fluorescence microscope is a wide-field fluorescence microscope. The wide-field fluorescence microscope is generally used for quantitative analysis of the image. As used in this manner, the term “wide-field” means that a system acquired 2-D image of (image size around 200 nm*200 nm or higher depending upon camera and magnification used in the system.

A sample illuminated with a wide-field fluorescence microscope is subject to uneven illumination deviating from a Gaussian profile of a light source. This uneven illumination is referred to as “roll-off”. The roll-off value can be expressed in the form of equation (I)

$$\text{Roll-off} = 100 \times \frac{( I max - I min )}{I max} \qquad ( I )$$

where Iₘₐₓ=maximum intensity detected at the center of the image of a homogeneously fluorescent sample and Iₘᵢₙ=minimum intensity detected at the diagonal corner of the image of the homogeneously fluorescent sample.

The method 200 of some embodiments comprises process 210 to determine the roll-off value for the fluorescence microscope. In some embodiments, the roll-off value of the fluorescence microscope is adjusted to be within a range from 65% to 60%, from 65% to 55%, from 65% to 50%, from 65% to 45%, from 65% to 40%, from 65% to 35%, from 65% to 30%, from 65% to 25%, from 65% to 20%, from 60% to 55%, from 60% to 50%, from 60% to 45%, from 60% to 40%, from 60% to 35%, from 60% to 30%, from 60% to 25%, from 60% to 20%, from 55% to 50%, from 55% to 45%, from 55% to 40%, from 55% to 35%, from 55% to 30%, from 55% to 25%, from 55% to 20%, from 50% to 45%, from 50% to 40%, from 50% to 35%, from 50% to 30%, from 50% to 25%, from 50% to 20%, from 45% to 40%, from 45% to 35%, from 45% to 30%, from 45% to 25%, from 45% to 20%, from 40% to 35%, from 40% to 30%, from 40% to 25%, from 40% to 20%, from 35% to 30%, from 35% to 25%, from 35% to 20%, from 30% to 25%, from 30% to 20%, or from 25% to 20%. In some embodiments, the roll-off value of the fluorescence microscope is less than or equal to 70%, 65%, 60%, 55%, 50%, 45%, 40%, 35%, 30%, 25%, 20%, 15%, 10% or 5%.

In one or more embodiments, the roll-off value is determined using a calibration slide. In some embodiments the calibration slide comprising a plurality of encapsulated fluorophores. In one or more embodiments, the roll-off value is determined by one or more of a fluorescent dye, a TetraSpeck™ bead (e.g., from Thermo Fisher Scientific), a fiducial marker or an Argolight calibration or standardization slide. In one or more embodiments, the roll-off value is determined by using an Argolight calibration slide. In some embodiments, the Argolight calibration slide is selected from at least one of a group consisting of ArgoPOWER™ slide, Argo-HM slide, Argo-SIM slide, Argo-LM slide, Argo-WP slide and Argo-CHECK slide.

In some embodiments, process 210 includes adjusting the roll-off value of the fluorescence microscope. In one or more embodiments, the roll-off value is adjusted by controlling the distance between the light source 120 and the excitation focus lens assembly 155.

The illumination power density can be estimated by imaging a standard test object containing a fluorophore probe to calibrate the system. The standard test object has a stable response and is within a depth of focus. The depth of focus is in a range of from 100 nm to 1000 nm, from 100 nm to 900 nm, from 100 nm to 800 nm, from 100 nm to 700 nm, from 100 nm to 600 nm, from 100 nm to 500 nm, from 100 nm to 400 nm, from 100 nm to 300 nm, from 100 nm to 200 nm, 200 nm to 1000 nm, from 200 nm to 900 nm, from 200 nm to 800 nm, from 200 nm to 700 nm, from 200 nm to 600 nm, from 200 nm to 500 nm, from 200 nm to 400 nm, from 200 nm to 300 nm, 300 nm to 1000 nm, from 300 nm to 900 nm, from 300 nm to 800 nm, from 300 nm to 700 nm, from 300 nm to 600 nm, from 300 nm to 500 nm, from 300 nm to 400 nm, 400 nm to 1000 nm, from 400 nm to 900 nm, from 400 nm to 800 nm, from 400 nm to 700 nm, from 400 nm to 600 nm, from 400 nm to 500 nm, 500 nm to 1000 nm, from 500 nm to 900 nm, from 500 nm to 800 nm, from 500 nm to 700 nm, or from 500 nm to 600 nm.

In some embodiments of the method 200, the sample is a multiplexed error-robust fluorescence in situ hybridization sample hybridized with a number of different fluorophore probes. In some embodiments, there are greater than or equal to two fluorophore probes. In some embodiments, the number of fluorophore probes is in a range of from 2 to 30, from 2 to 25, from 2 to 20, from 2 to 16, from 4 to 20, from 4 to 16, from 6 to 20, from 6 to 16, from 8 to 20, from 8 to 16, from 10 to 20 or from 10 to 16. In some embodiments, the sample is prepared from a cell line. In some embodiments, the cell line comprises epithelial lung cells. In some embodiments, the cell line comprises A549 cells. The skilled artisan will recognize that other cell lines can be used and are within the scope of the disclosure.

In some embodiments, each selected fluorophore probe has a unique excitation wavelength. Leaving all components of the fluorescence microscope unchanged. Referring to FIG. 1, in one or more embodiments, the fluorescence microscope comprises a stage 111 configured to support the sample flow cell 109. The stage 111 of some embodiments is connected to a focal plane actuator 112 configured to move the stage 111 perpendicular to the focal plane. In some embodiments, the focal plane actuator 112 is further configured to move the stage 111 parallel to the focal plane. In some embodiments, the focal plane actuator 112 is connected to a focal plane controller or a focal plane processor. The focal plane controller or the focal plane processor of some embodiments is configured to track a fluorescing object in the sample.

At process 220, an excitation wavelength and an emission wavelength are selected for a chosen fluorophore probe. At process 230, the sample is hybridized with the first fluorophore probe associated with the excitation and emission wavelengths chosen in process 220. The skilled artisan will recognize that either process 220 or process 230 can be performed first or can be performed at the same time.

In one or more embodiments, the stage further comprises a flow-cell assembly. The flow-cell comprises a flow-cell holder configured to hold the sample, an inlet connected to one or more reservoirs, and an outlet connected to a discard container. The reservoirs comprising one or more wash buffer reservoirs or fluorophore probe reservoirs. In one or more embodiments, the flow-cell assembly is connected to a flow-cell assembly controller or a flow-cell assembly processor, that is configured to flow a fluorophore probe or wash buffer from the fluorophore probe reservoir or the wash buffer reservoir respectively to the sample in a predetermined quantity, incubate the sample with the fluorophore probe for a predetermined time, wash unbound fluorophore probes with the wash buffer, and discard the unbound fluorophore probes into the discard container. The pre-determined quantity is in the range of from 1 μl to 50 ml, from 1 ml to 50 ml, from 5 ml to 50 ml, from 10 ml to 50 ml, from 1 ml to 20 ml, from 5 ml to 20 ml, or from 10 ml to 20 ml. The predetermined time is in the range of from 1 s to 6 hours, 1 min to 6 hours, 5 min to 6 hour, 20 min to 6 hour, 30 min to 6 hour, 1 hour to 6 hour, 2 hour to 6 hour, 4 hour to 6 hour, 1 min to 60 min, 5 min to 60 min, 10 min to 60 min, 20 min to 60 min, 20 min to 60 min, or 30 min to 60 min.

After hybridization of the sample in process 230, an image of the sample is acquired in process 240. In one or more embodiments, the method 200 further comprises process 250 in which the sample image is quantitatively analyzed to determine a number and spatial distribution of fluorescing first fluorophore probes in the image.

After analysis, or while the data analysis is being performed, decision point 260 is considered. If image data has been captured for a sufficient number of fluorophore probes, the method 200 proceeds to process 270 in which the quantitative analysis of the images are combined. If image data has not been captured for a predetermined number of fluorophore probes, the method 200 moves to process 280 in which the sample is photobleached to deactivate the hybridized fluorophore probe. In one or more embodiments, the method 200 of fluorescence image acquisition further comprises photobleaching the sample to deactivate the fluorophore probes.

After photobleaching in process 280, the method 200 repeats processes 220-250 using a second fluorophore probe. In some embodiments, the method 200 further comprises selecting a second excitation wavelength and a second emission wavelength for a second fluorophore probe in a repeat of process 220. The fluorescence microscope in some embodiments is adjusted to a second focal plane different than the first focal plane, the second focal plane based on the second excitation wavelength. The sample is hybridized in process 230 with the second fluorophore probe, and a second image of fluorescently emitted light from the sample hybridized with the second fluorophore probe is acquired at process 240. The second excitation wavelength is different from the first excitation wavelength. In some embodiments, the method further comprises quantitatively analyzing the second image at process 250 to determine a second number and a second spatial distribution of fluorescing second fluorophore probes in the second image.

In one or more embodiments, the method further comprises process 270 where the quantitative analyses of the fluorescing first fluorophore probes and fluorescing second fluorophore probes are combined to determine a copy number and spatial distribution of RNA in the sample.

In some embodiments, the method further comprises repeating the process 280, 220, 230, 240 and 250 for one or more additional fluorophore probes. In some embodiments, the method 200 further comprises selecting one or more additional excitation wavelength and one or more additional emission wavelength for one or more additional fluorophore probes, adjusting the fluorescence microscope to a focal plane based on the one or more additional excitation wavelength, hybridizing the sample with one of the one or more additional fluorophore probes, acquiring one or more additional image of fluorescently emitted light from the sample hybridized with the one or more additional fluorophore probes, quantitatively analyzing the one or more additional images to determine an additional number and additional spatial distribution of the one or more additional fluorescing fluorophore probes, and combining the quantitative analysis of the fluorescing fluorophore probes to determine a copy number and spatial distribution of RNA in the sample.

Another aspect described in the disclosure includes a method of fluorescence image acquisition. In one or more embodiments, the method comprises determining a roll-off value for a fluorescence microscope, adjusting the roll-off value of the fluorescence microscope in the range of from 31% to 65%, the roll-off value adjusted by controlling the distance between the light source and the excitation focus lens assembly, and quantitatively analyzing a sample using a plurality of fluorophore probes to generate a spatial distribution for each fluorophore probe.

In one or more embodiments, the method of quantitatively analyzing the sample comprises selecting a fluorophore probe comprising a nucleotide sequence and a fluorophore, selecting an excitation wavelength and emission wavelength for the fluorophore probe, hybridizing the sample with the fluorophore probe, and acquiring an image of fluorescently emitted light from the hybridized sample using the fluorescence microscope. In some embodiments, the method further comprises photobleaching the sample after acquiring an image of the fluorescently emitted light from the hybridized sample prior to hybridizing the sample with a different fluorophore probe.

Another aspect of the disclosure provides a fluorescence microscope with a sample stage configured to hold a sample, a variable wavelength excitation directed at the sample stage, a detector configured to detect light emitted from the sample, and a controller configured to adjust a roll-off value for the microscope to be in the range of 31% to 65%.

The processes described herein may generally be stored in the memory as a software routine that, when executed by a controller or processor, standardizes one or more parameter of a fluorescence microscope as described in the present disclosure. The software routine may also be stored and/or executed by a second controller or processor (not shown) that is remotely located from the hardware being controlled by the controller or processor. Some or all of the method of the present disclosure may also be performed in hardware. As such, the process may be implemented in software and executed using a computer system, in hardware as, e.g., an application specific integrated circuit or other type of hardware implementation, or as a combination of software and hardware. The software routine, when executed by the processor or controller, transforms the general purpose computer into a specific purpose computer (controller) that controls the fluorescence microscope operation such that the processes are performed. The processes may be stored on a non-transitory computer readable medium including instructions, that, when executed by a controller of a fluorescence microscope, causes a fluorescence microscope to perform one or more of the methods described herein.

Referring back to FIG. 1, in some embodiments, at least one controller 170 is coupled to one or more of the flow cell assembly 110, light source 120 or detector assembly 160. In some embodiments, there are more than one controller 170 connected to the flow cell assembly 110, light source 120 or detector assembly 160. In some embodiments, the at least one control 170 is connected to the optics (e.g., lenses, filters, reflectors), focal plane actuator 112 or other components.

The controller 170 may be one of any form of general-purpose computer processor, microcontroller, microprocessor, etc., that can be used in an industrial setting for controlling various chambers and sub-processors. The at least one controller 170 can have a processor 172, a memory 174 coupled to the processor 172, input/output devices 176 coupled to the processor 172, and support circuits 178 to communicate between the different electronic components. The memory 174 can include one or more of transitory memory (e.g., random access memory) and non-transitory memory (e.g., storage).

The memory 174, or computer-readable medium, of the processor may be one or more of readily available memory such as random access memory (RAM), read-only memory (ROM), floppy disk, hard disk, or any other form of digital storage, local or remote. The memory 174 can retain an instruction set that is operable by the processor 172 to control parameters and components of the system 100. The support circuits 178 are coupled to the processor 172 for supporting the processor in a conventional manner. Circuits may include, for example, cache, power supplies, clock circuits, input/output circuitry, subsystems, and the like.

Processes may generally be stored in the memory as a software routine that, when executed by the processor, causes the process chamber to perform processes of the present disclosure. The software routine may also be stored and/or executed by a second processor (not shown) that is remotely located from the hardware being controlled. Some or all of the method of the present disclosure may also be performed in hardware. As such, the process may be implemented in software and executed using a computer system, in hardware as, e.g., an application specific integrated circuit or other type of hardware implementation, or as a combination of software and hardware. The software routine, when executed by the processor, transforms the general purpose computer into a specific purpose computer (controller) that controls the chamber operation such that the processes are performed.

In some embodiments, the controller 170 has one or more configurations to execute individual processes or sub-processes to perform the method. The controller 170 can be connected to and configured to operate intermediate components to perform the functions of the methods. For example, the controller 170 can be connected to and configured to control one or more of pumps, gas valves, actuators, motors, flow controllers, mirrors, filters, lenses, bandpass filter apertures, lights sources or power supplies.

The controller 170 of some embodiments has one or more configurations selected from: a configuration to flow a fluorophore probe from a reservoir into a sample flow cell; a configuration to flow a wash buffer from a wash buffer reservoir to a sample flow cell; a configuration to acquire an image of the sample flow cell using a detector; a configuration to analyze the image of the sample flow cell to determine the spatial distribution of fluorescing fluorophore probes in the sample flow cell; a configuration to analyze the image of the sample flow cell to quantify the amount of fluorescing fluorophore probes in the sample flow cell; a configuration to adjust the focal plane of the microscope optics; a configuration to adjust the position of the stage supporting the sample flow cell; a configuration to photobleach the sample flow cell; a configuration to open an aperture of the object lens and/or aperture of the objective lens; a configuration to control the power of a laser to photobleach the sample flow cell; a configuration to adjust the roll-off value of the fluorescence microscope; and/or a configuration to adjust the power density of the fluorescence microscope.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

What is claimed is:

1. A method of fluorescence image acquisition, the method comprising:
determining a roll-off value for a fluorescence microscope, the fluorescence microscope comprising a light source and an excitation focus lens assembly;
adjusting the roll-off value of the fluorescence microscope to be within a range of 31% to 65%;
selecting an first excitation wavelength and first emission wavelength for a first fluorophore probe;
hybridizing a sample with the first fluorophore probe; and
acquiring an image of the sample using the fluorescence microscope configured with a first focal plane, the image comprising fluorescently emitted light from the sample.

2. The method of claim 1, wherein the sample is a multiplexed error-robust fluorescence in-situ hybridization sample hybridized with a number of different fluorophore probes, the number greater than or equal to two.

3. The method of claim 2, wherein the number of different fluorophore probes are in the range of six to twenty.

4. The method of claim 2, wherein the sample is prepared from A549 Cell line.

5. The method of claim 1, wherein the fluorescence microscope is a wide-field fluorescence microscope.

6. The method of claim 1, wherein the roll-off value is determined using a calibration slide comprising a plurality of encapsulated fluorophores.

7. The method of claim 1, wherein the roll-off value is adjusted by controlling a distance between the light source and the excitation focus lens assembly.

8. The method of claim 2, further comprising quantitatively analyzing the image to determine a number and spatial distribution of fluorescing first fluorophore probes in the image.

9. The method of claim 8, further comprising photobleaching the sample to deactivate the fluorophore probes.

10. The method of claim 9, further comprising:
selecting a second excitation wavelength and second emission wavelength for a second fluorophore probe;
adjusting the fluorescence microscope to a second focal plane different than the first focal plane, the second focal plane based on the second excitation wavelength;
hybridizing the sample with the second fluorophore probe; and
acquiring a second image of fluorescently emitted light from the sample hybridized with the second fluorophore probe.

11. The method of claim 10, further comprising quantitatively analyzing the second image to determine a second number and second spatial distribution of fluorescing second fluorophore probes in the second image.

12. The method of claim 11, further comprising combining the quantitative analysis of the fluorescing first fluorophore probes and fluorescing second fluorophore probes to determine a copy number and spatial distribution of RNA in the sample.

13. The method of claim 11, further comprising:
selecting one or more additional excitation wavelength and one or more additional emission wavelength for one or more additional fluorophore probes;
adjusting the fluorescence microscope to a focal plane based on the one or more additional excitation wavelength;
hybridizing the sample with one of the one or more additional fluorophore probes;
acquiring one or more additional image of fluorescently emitted light from the sample hybridized with the one or more additional fluorophore probes;
quantitatively analyzing the one or more additional images to determine an additional number and additional spatial distribution of the one or more additional fluorescing fluorophore probes; and
combining the quantitative analysis of the fluorescing fluorophore probes to determine a copy number and spatial distribution of RNA in the sample.

14. A method of fluorescence image acquisition, the method comprising:
determining a roll-off value for a fluorescence microscope, the fluorescence microscope comprises a light source and an excitation focus lens assembly;
adjusting the roll-off value of the fluorescence microscope to be within a range of 31% to 65%, the roll-off value adjusted by controlling a distance between the light source and the excitation focus lens assembly; and
quantitatively analyzing a sample using a plurality of fluorophore probes to generate a spatial distribution for each fluorophore probe.

15. The method of claim 14, wherein the roll-off value is determined using a calibration slide comprising a plurality of encapsulated fluorophores.

16. The method of claim 15, wherein the calibration slide is selected from the group consisting of Argo POWER™ calibration slide, Argo-HM calibration slide, Argo-SIM calibration slide, Argo-LM calibration slide, Argo-WP calibration slide and ArgoCheck calibration slide

17. The method of claim 15, wherein quantitatively analyzing the sample comprising:
selecting a fluorophore probe comprising a nucleotide sequence and a fluorophore;
selecting an excitation wavelength and emission wavelength for the fluorophore probe;
hybridizing the sample with the fluorophore probe; and
acquiring an image of fluorescently emitted light from the hybridized sample using the fluorescence microscope.

18. The method of claim 17, wherein quantitatively analyzing the sample further comprises photobleaching the sample after acquiring an image of the fluorescently emitted light from the hybridized sample prior to hybridizing the sample with a different fluorophore probe.

19. A fluorescence microscope comprising:
a sample stage configured to hold a sample;
a variable wavelength excitation light source directed at the sample stage;
an excitation focus lens assembly configured to focus a light from the light source on the sample;
a detector configured to detect light emitted from the sample; and
a controller configured to adjust a roll-off value for the microscope to be in the range of 31% to 65%.

20. The fluorescence microscope of claim 19, wherein the controller is configured to control a distance between the light source and the excitation focus lens assembly.
